(19) United States
(12) Patent Application Publication
(10) Pub. No.: US 2024/0336984 A1
(43) Pub. Date: Oct. 10, 2024

(54) NUCLEIC ACID DETECTION

(71) Applicant: Llusern Scientific Ltd, Cardiffs (GB)

(72) Inventors: Jeroen NIEUWLAND, Pontypridd (GB); Ali ROULA, Pontypridd (GB); Emma HAYHURST, Pontypridd (GB)

(21) Appl. No.: 18/558,490

(22) PCT Filed: May 12, 2022
(86) PCT No.: PCT/GB2022/051205

§ 371 Date: Nov. 1, 2023

(30) Foreign Application Priority Data
May 14, 2021 (GB) 2106891.1

(51) Int. Cl. C12Q 1/70; B01L 3/00; B01L 7/00; C12Q 1/6851

(52) U.S. Cl. CPC C12Q 1/701; B01L 3/502715; B01L 7/52; C12Q 1/6851; B01L 2200/16; B01L 2300/0663; B01L 2300/0681; B01L 2300/1805

(57) ABSTRACT

Disclosed herein is a method of detecting a target nucleic acid in a biological sample, the method comprising: i) combining the sample with reagents to enable amplification of the target nucleic acid and fluorescent labelling of any amplified product; ii) detecting fluorescence, and optionally determining that the baseline fluorescence level of the amplification reaction does not exceed a predetermined threshold; and iii) determining whether the target nucleic acid is present in the sample based on one or more of the following parameters: a) time to start of reaction, signalled by an increase in fluorescence; b) peak fluorescence intensity.

### TECHNICAL FIELD

The present invention relates to a method of detecting presence or absence of a target nucleic acid in a biological sample and a point-of-care device for detecting a target nucleic acid in a biological sample.

### BACKGROUND

Nucleic acid amplification techniques such as polymerase chain reaction (PCR) and loop-mediated isothermal amplification (LAMP) are used for detection of target nucleic acids in biological samples. Reverse transcription loop-mediated isothermal amplification (RT-LAMP) combines LAMP with a reverse transcription step to allow the detection of RNA.

PCR is possibly the most widely used method but has the disadvantage of requiring costly laboratory instrumentation due to the need for multiple temperature changes for each amplification cycle and highly skilled operators as well as often taking days to provide results. Isothermal nucleic acid amplification assays such as LAMP provide a rapid cost-effective alternative to PCR. However, there remains the need for increased specificity of these tests by reducing false positive signals due to issues such as primer-dimer formation. Development of cost effective and reliable test devices and kits also remains important

There is, therefore, a continuing need for accurate, rapid, convenient and cost-effective diagnostic testing solutions for the detection of infectious agents such as SARS-CoV-2 in point of care settings.

### SUMMARY

According to a first aspect of the present invention, there is provided a method of detecting a target nucleic acid in a biological sample, the method comprising:
i) combining the sample with reagents to enable amplification of the target nucleic acid and fluorescent labelling of any amplified product;
ii) detecting fluorescence, and optionally determining that the baseline fluorescence level of the amplification reaction does not exceed a predetermined threshold; and
iii) determining whether the target nucleic acid is present in the sample based on one or more of the following parameters:
   a) time to start of reaction, signalled by an increase in fluorescence; and
   b) peak fluorescence intensity.

A second aspect of the invention provides a device for detecting a target nucleic acid in a biological sample, the device comprising;
a reaction chamber, which in use receives the sample and reagents for performing amplification and fluorescent labelling of the amplification product;
a light source which emits light at a wavelength corresponding to an excitation wavelength of the fluorescent label;
a detector, for detecting fluorescence; and
a user interface;
wherein the device is programmed to classify the biological sample as positive, negative or undetermined for the presence of the target nucleic and present the classification on the user interface, the classification being based on one or more of the following parameters:
   a) baseline fluorescence level;
   b) time to start of reaction, signalled by an increase in fluorescence; and
   c) peak fluorescence intensity.

A third aspect of the invention provides a device for detecting a target nucleic acid in a biological sample, the device comprising;
a reaction chamber, which in use receives the sample and reagents for performing amplification and fluorescent labelling of the amplification product;
a light source which emits light at a wavelength corresponding to an excitation wavelength of the fluorescent label;
a detector, for detecting fluorescence; and
a user interface, which indicates whether the target nucleic acid is present in the sample;
wherein the device further comprises a filter between the reaction chamber and detector, which filters light at wavelengths emitted by the light source, and wherein there is no direct line of sight between the light source and detector.

A fourth aspect of the invention provides a kit comprising a swab for sample collection, and an assay solution comprising reagents for performing amplification and fluorescent labelling of the amplification product;
the swab comprising a handle, a sample collection tip, and a collar between the handle and the sample collection tip;
wherein the collar has a larger diameter than the opening of a standard 0.2 mL PCR tube;
and wherein the tip is formed from polypropylene and comprises a plurality of circumferential ribs configured to collect a predetermined volume of liquid sample by capillary action and adhesion.

Features described in relation to one aspect are hereby explicitly disclosed in combination with the other aspects, to the extent that they are combinable. In particular, features described in relation to one of the second and third aspects are explicitly disclosed in combination with the other aspect.

Further features and advantages of the invention will become apparent from the following description of preferred embodiments of the invention, given by way of example only, which is made with reference to the accompanying drawings.

### BRIEF DESCRIPTION OF THE DRAWINGS

FIG. 1 is a schematic illustration of a process according to the invention by which determination as to the presence or absence of the target nucleic acid may be completed.

FIG. 2 shows a heating block which forms part of a device according to the invention.

FIGS. 3A and 3B shows a device according to the invention.

FIGS. 4 and 5 each show fluorescence data obtained by a device according to the invention.

FIG. 6 shows a swab used to collect oral-nasal samples for processing according to the inventive method described herein.

FIG. 7 shows a schematic arrangement of some components of a device according to the invention.

FIG. 8 shows fluorescence data obtained by a device according to the invention.

### DETAILED DESCRIPTION

The present invention provides rapid, point-of-care detection of a target nucleic acids in a biological sample with improved accuracy. Such detection can be used to diagnose infection and disease. In some cases, the target nucleic acid may be from a microorganism. In some cases, the target nucleic acid may be from an infection marker in a human or animal.

The present invention provides a method for detecting the presence of absence of a target nucleic acid in a biological sample based on an amplification reaction, with fluorescent labelling, and detection of fluorescence. The inventors have identified several parameters of the resulting fluorescence curve which may be used, individually or in combination, to determine whether the target nucleic acid is present or absent. These parameters are used to reduce the incidence of false positive signals, such as due to primer-dimer formation, thereby improving the specificity of nucleic acid detection.

One parameter identified by the inventors which may inform determination is the baseline fluorescence level. If the baseline fluorescence level (i.e. the fluorescence level before the amplification reaction begins) exceeds 50% of the detection limit of the device, no determination as to presence or absence of the nucleic acid is possible. In such a scenario, the sample is classified as undetermined and the detection method will need to be carried out again to determine the presence or absence of target nucleic acid in that sample. In some cases, twice the inherent fluorescence of the dye is used as the threshold, which if exceed by baseline means that no determination is possible, rather than 50% of the detection limit of the device.

Another parameter identified by the inventors which may inform determination as to presence or absence of the target nucleic acid is the time to start of amplification reaction, signalled by an increase in fluorescence. The inventors have found that amplification of the target nucleic acid occurs within a set time window after combination of the sample and amplification reagents, and incubation under reaction conditions. In some cases, the method involves monitoring fluorescence over time to establish when fluorescence starts to increase relative to the time when the sample and reagents are combined.

In some cases, the time window is defined by a predetermined minimum time value (measured from the time of combination of the sample and reagents). In some cases, if an increase in fluorescence begins before this minimum time value, no positive determination as to the presence of the target nucleic acid is possible.

In some cases, the time window is defined by predetermined minimum and maximum time values (measured from the time of combination of the sample and reagents). In some cases, if an increase in fluorescence begins outside of a predetermined time window, no positive determination as to the presence of the target nucleic acid is possible.

If the increase in fluorescence starts outside of the set time window, this may be an indication that the fluorescence is caused by primer-dimer amplification, and no determination as to presence or absence of the nucleic acid is possible.

In some cases, if the increase begins before the predetermined time window, no determination is possible, and if the increase begins after the predetermined time window, the determination is “negative” (i.e. target nucleic acid is not present).

In some cases, if time to start of reaction is between 8 and 18 minutes, such as from about 8, 9, 10, 11, 12 or 13 minutes to about 15, 16, 17 or 18 minutes, the determination is positive. In some cases, if time to start of reaction is less than 8 minutes, the sample is classified as undetermined.

Another parameter identified by the inventors which may inform determination as to presence or absence of the target nucleic acid is the peak fluorescence intensity. The inventors have found that amplification of the target nucleic acid occurs to reliably increase fluorescence by a threshold amount. If the increase in fluorescence does not exceed this threshold, this may be an indication that the fluorescence is caused by primer-dimer amplification, and the determination is negative (i.e. the target nucleic acid is absent). In some cases, if peak fluorescence>1.5*baseline fluorescence, decision is “present”, otherwise “absent”. In some cases, the threshold is 1.6*baseline fluorescence, 1.7*baseline fluorescence or 1.8*baseline fluorescence.

In some cases, the method of determining whether the target nucleic acid is present or absent is based on time to start of reaction and peak fluorescence intensity. In some cases, the method further includes determining that the baseline fluorescence level of the amplification reaction does not exceed a predetermined threshold.

In one example, the method of determining whether the target nucleic acid is present or absent includes:
a) measuring the baseline fluorescence level and
   i) if this level exceeds a predetermined threshold, classifying the sample as undetermined and carrying out the method again;
   ii) if this level does not exceed a predetermined threshold, proceed to step b);
b) measuring the peak fluorescence intensity and
   i) if the peak fluorescence intensity does not exceed a predetermined threshold, classifying the sample as negative;
   ii) if the peak fluorescence intensity exceeds a predetermined threshold, proceed to step c);
c) measuring the time to start of amplification reaction, signalled by an increase in fluorescence and
   i) if the increase in fluorescence starts within a predetermined time window, the determination is positive (i.e. the target nucleic acid is present)
   ii) if the increase in fluorescence starts outside of this time window, classifying the sample as undetermined and carrying out the method again.

In some cases, the biological sample has undergone no processing between sample collection and the start of the above determination method. That is, the above method can be practised on direct biological samples, without any need for refinement of the sample such as purification or other pre-processing steps.

DNA or RNA from a biological sample may be detected by the method and device described herein. In some such cases, where RNA is to be detected, the sample is contacted with reverse transcriptase prior to amplification to provide a double stranded DNA target nucleic acid for amplification.

In some cases, the amplification is an isothermal amplification reaction. In some such cases, the reaction is a loop-mediated isothermal amplification (LAMP) and the method comprises contacting the sample with a set of LAMP primers specific for the target nucleic acid. LAMP amplifies DNA rapidly under isothermal conditions by using a DNA polymerase with high displacement strand activity and a set of specifically designed primers to amplify targeted DNA strands. Following its first discovery by Notomi et al. (Nucleic Acids Res 28: E63), LAMP has been further developed over the years for the detection of infectious diseases caused by micro-organisms in humans, livestock and plants. A set of four (or six) different primers binds to six (or eight) different regions on the target gene making it highly specific (Notomi et al. 2000). This primer set consists of two outer (F3 and B3) primers, two inner primers (forward inner primer (FIP) and backward inner primer (BIP)) and loop primers (loop forward and loop backward).

In some cases, the microorganism is a bacterium. In some such cases, the bacterium is Escherichia coli, Clostridium or Streptococcus. The bacterium being detected may be Mycobacterium tuberculosis, Streptococcus pneumoniae, Bordetella pertussis, Klebsiella pneumoniae, Salmonella typhi, Campylobacter jejuni, Campylobacter coli, Helicobacter pylori or Escherichia coli. In some cases, the microorganism is a virus. In some such cases, the virus is selected from the group consisting of an influenza virus, a coronavirus, respiratory syncytial virus, adenovirus, varicella zoster, cytomegalovirus and Hepatoviruses such as hepatitis B.

In some case, the target nucleic acid is part of the hRSV RNA genome. Detection of hRSV A and B groups using LAMP assay can use reagents as described in Hu et al. Sensitive fluorescent LAMP for rapid RSV detection; J Infect Dev Ctries 2019; 13(12):1135-1141. The amplification of hRSV RNA may be monitored in real time using SYTO-9 as the fluorescent dye.

In some cases, the target nucleic acid is part of the SARS-CoV-2 RNA genome, and the method can thus be used to diagnose a COVID-19 infection. In some particular cases, the amplification reaction specifically amplifies the E1 and/or N2 targets in the SARS-CoV-2 RNA genome.

In some cases, the amplification is a loop-mediated isothermal amplification (LAMP) reaction and wherein the set of LAMP primers comprises six primers to specifically amplify the E1 and/or N2 targets in the SARS-CoV-2 RNA genome.

In some cases, the set of LAMP primers comprises six primers for the detection of E1 having nucleic acid sequence at least 95% identical to SEQ ID NOs: 1-6. In some cases, the six primers for the detection of E1 are identical to SEQ ID NOs: 1-6.

| | |
|---|---|
| SEQ ID No. 1 | TGAGTACGAACTTATGTACTCAT |
| SEQ ID No. 2 | TTCAGATTTTTAACACGAGAGT |
| SEQ ID No. 3 | ACCACGAAAGCAAGAAAAAGAAGT-TCGTTTCGGAAG |
| | AGACAG |
| SEQ ID No. 4 | TTGCTAGTTACACTAGCCATCCTT-AGGTTTTACAAG |
| | ACTCACGT |
| SEQ ID No. 5 | CGCTATTAACTATTAACG |
| SEQ ID No. 6 | GCGCTTCGATTGTGTGCGT |

In some cases, the set of LAMP primers comprises six primers for the detection of N2 having nucleic acid sequence at least 95% identical to SEQ ID NOs: 7-12. In some cases, the six primers for the detection of N2 are identical to SEQ ID NOs: 7-12.

| | |
|---|---|
| SEQ ID No. 7 | ACCAGGAACTAATCAGACAAG |
| SEQ ID No. 8 | GACTTGATCTTTGAAATTTGGATCT |
| SEQ ID No. 9 | TTCCGAAGAACGCTGAAGCGGAACTGATTACAAACA |
| | TTGGCC |
| SEQ ID No. 10 | CGCATTGGCATGGAAGTCACAATTTGATGGCACCTG |
| | TGTA |
| SEQ ID No. 11 | GGGGGCAAATTGTGCAATTTG |
| SEQ ID No. 12 | CTTCGGGAACGTGGTTGACC |

In some cases, the reaction mixture for the LAMP reaction targeting the E1 and/or N2 targets in the SARS-CoV-2 RNA genome further comprises guanidine hydrochloride. In some cases, the LAMP reaction is as described by Zhang et al, Biotechniques, vol. 69, no. 3, 2020 (https.//doi.org/10.2144/btn-2020-0078), “Enhancing colorimetric loop-mediated isothermal amplification speed and sensitivity with guanidine chloride” which is herein incorporated by reference in its entirety except that in place of colorimetric detection a fluorescent dye, such as SYTO 9, was used for real time monitoring of the amplification. Other suitable dyes which are compatible with the apparatus and method described herein include SYBR Green I, LC Green, LC Green Plus+, EvaGreen, FAM, Fluorescein and Alexa Fluor 488.

Table 1 below provides the materials and reagents along with the stock concentration for an exemplary LAMP reaction targeting the E1 and/or N2 targets in the SARS-CoV-2 RNA genome.

TABLE 1

| Stock | |
|---|---|
| Concentration | Reagent |
| 100% | Nuclease-free molecular grade water |
| 10x | Reaction buffer from Bst kit |
| 100 mM | MgSO4 from Bst kit |
| 10 mM | dNTPs |
| 5 mM | SYTO-9 |
| 6M | Guanidine HCL |
| 100 μM | E1 FIP |
| | ACCACGAAAGCAAGAAAAAGAAGTTCGTTTCGGAAG |
| | AGACAG |
| 100 μM | E1 BIP |
| | TTGCTAGTTACACTAGCCATCCTTAGGTTTTACAAG |
| | ACTCACGT |
| 100 μM | E1 F3 |
| | TGAGTACGAACTTATGTACTCAT |
| 100 μM | E1 B3 |
| | TTCAGATTITTAACACGAGAGT |
| 100 μM | E1 LoopF |
| | GCGCTTCGATTGTGTGCGT |
| 100 μM | E1 LoopB |
| | CGCTATTAACTATTAACG |
| 100 μM | N2 FIP |
| | TTCCGAAGAACGCTGAAGCGGAACTGATTACAAACA |
| | TTGGCC |
| 100 μM | N2 BIP |
| | CGCATTGGCATGGAAGTCACAATTTGATGGCACCTG |
| | TGTA |
| 100 μM | N2 F3 |
| | ACCAGGAACTAATCAGACAAG |
| 100 μM | N2 B3 |
| | GACTTGATCTTTGAAATTTGGATCT |
| 100 μM | N2 LoopF |
| | GGGGGCAAATTGTGCAATTTG |
| 100 μM | N2 LoopB |
| | CTTCGGGAACGTGGTTGACC |
| 8000 U/ml | Bst 2.0 Warmstart |
| 15,000 U/ml | RTx Warmstart |

When using the above-described assay to test for SARS-CoV-2, the following parameters and threshold values may be used to determine presence or absence of the microorganism:
If time to start of reaction<8 minutes, decision is “undetermined”. If >8 minutes, proceed to:
If peak fluorescence>1.8*baseline fluorescence, decision is “present”, otherwise “absent”.

In some cases, the sample is an oral-nasal sample, or a saliva sample.

In some cases, the method further comprises an initial stage of collecting the sample from a patient. In some cases, there are no further processing steps completed on the sample before the determination method is commenced.

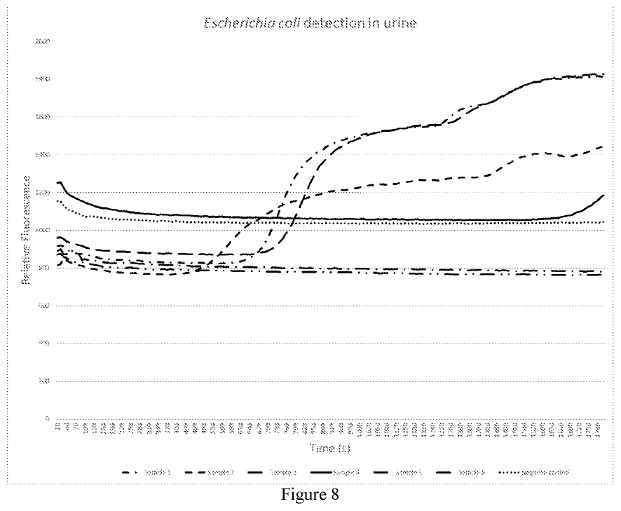

In some cases, the target nucleic acid is DNA from Escherichia coli, and the method can thus be used to diagnose an e-coli infection. In some such cases, the sample is a urine sample. RT-LAMP is performed using appropriate primers. Data is illustrated in FIG. 8. In this case, it can be seen an increase in fluorescence is observed for samples 1, 2, 3 and 4.
For samples 1, 2 and 3, the algorithm returns a ‘positive’ outcome.
However, the fluorescence level for sample 4 begins to rise too late, and the algorithm returns an “negative” outcome.
   The samples in this experiment were also subjected to standard culture testing by Public Health Wales and the outcomes were positive for samples 1-3, and negative for samples 4-6. It can thus be seen that the method described herein has returned correct clinical diagnoses for samples 1-6.

The same algorithm parameter values may be used for the e-coli and covid-19 systems. However, in some cases, the thresholds selected may be changed dependent on the assay and target nucleic acid.

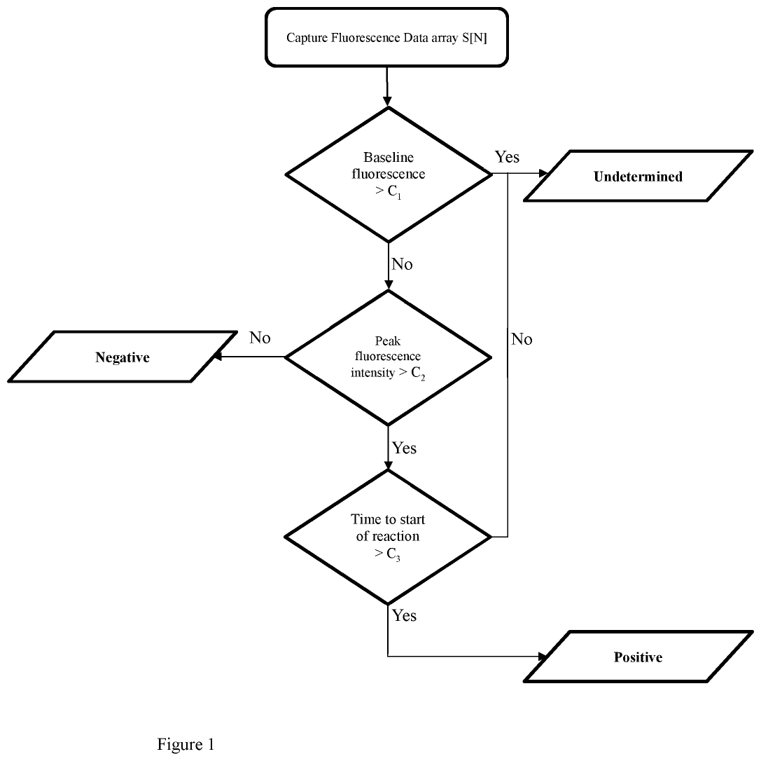

FIG. 1 schematically illustrates a process by which determination as to the presence or absence of the target nucleic acid is undertaken. Parameters C₁, C₂ and C₃ are determined experimentally. In an alternative embodiment, the final step may read “Time to start of reaction C₄>t>C₃”, defining a time window with maximum and minimum time values (with time measured relative to time of combination of sample and reagents).

The invention also provides a device for detecting a target nucleic acid in a biological sample, the device comprising;
a reaction chamber, which in use receives the sample and reagents for performing amplification and fluorescent labelling of the amplification product;
a light source which emits light at a wavelength corresponding to an excitation wavelength of the fluorescent label;
a detector, for detecting fluorescence; and
a user interface;
wherein the device is programmed to classify the biological sample as positive, negative or undetermined for the presence of the target nucleic and present the classification on the user interface, the classification being based on one or more of the following parameters:
   a) baseline fluorescence level;
   b) time to start of reaction, signalled by an increase in fluorescence; and
   c) peak fluorescence intensity.

In other words, the invention provides a device for performing the method described herein. Features disclosed in relation to the method are hereby specifically disclosed in combination with the device, and vice versa. In some cases, the device may be a point-of-care device.

In some cases, there is no direct line-of-sight between the light source and detector. Suitably, in some cases a straight line from the light source to the reaction chamber is perpendicular to a straight line from the reaction chamber to the detector. In some cases, the device further comprises a filter between the reaction chamber and detector, which filters light at wavelengths emitted by the light source. These features reduce incidence of light from the light source reaching the detector, and thus improve sensitivity to fluorescent light emitted by the amplification products.

In some cases, the reaction chamber comprises a well in a heating block, and wherein an insulative layer at least partially surrounds the heating block. In some such cases, the heating block includes a plurality of resistors which heat the block on application of a current by a control unit. In some such cases, the heating block includes a plurality of temperature sensors which feedback into the control unit.

The invention also provides a device for detecting a target nucleic acid in a biological sample, the device comprising;
a reaction chamber, which in use receives the sample and reagents for performing amplification and fluorescent labelling of the target nucleic acid;
a light source which emits light at a wavelength corresponding to an excitation wavelength of the fluorescent label;
a detector, for detecting fluorescence; and
a user interface, which indicates whether the target nucleic acid is present in the sample;
wherein the device further comprises a filter between the reaction chamber and detector, which filters light at wavelengths emitted by the light source, and wherein there is no direct line-of-sight between the light source and detector.

In some cases, a straight line from the light source to the reaction chamber is perpendicular to a straight line from the reaction chamber to the detector.

In some cases, the device can be operated with a 12V power source, such as a battery. This means that the device is more readily portable.

In some cases, the device is programmed to perform the method described herein. Features disclosed in relation to the method are hereby specifically disclosed in combination with the device, and vice versa. In some cases, the device may be a point-of-care device.

In some cases, the reaction chamber comprises a well in a heating block, and wherein an insulative layer at least partially surrounds the heating block. In some such cases, the heating block includes a plurality of resistors which heat the block on application of a current by a control unit. In some such cases, the heating block includes a plurality of temperature sensors which feedback into the control unit.

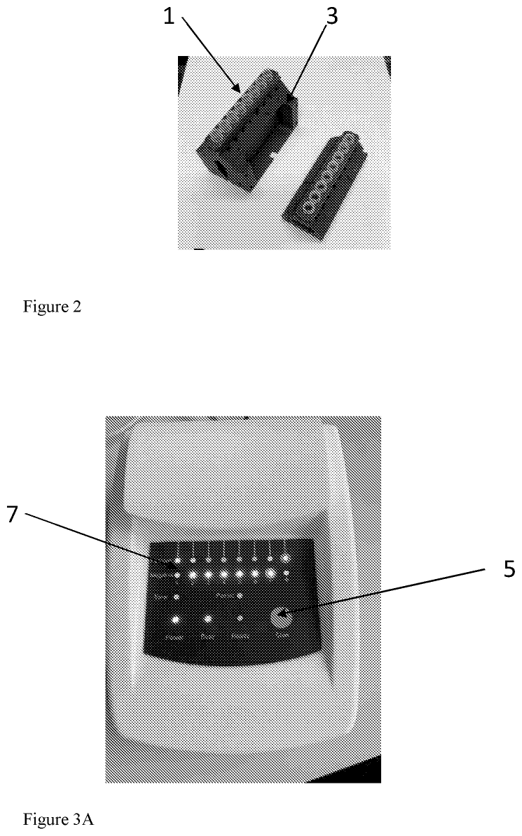

In an example device, with reference to FIG. 2, eight wells 1 are provided in a heating block 3. The heating block is formed from aluminium and is engineered to provide the best trade-off between temperature stability and small size (facilitating portability and ensuring low cost). The shape of and volume of the aluminium block allows maximum temperature diffusion to the sample wells while minimising heat transfer to the excitation LEDs/photodetector circuitry. Two independent temperature sensors feed into a PID controller to ensure the target temperature is reached swiftly without significant overshoot or oscillation.

In use, two of the wells 1 are loaded with control samples; one negative control and one positive. Up to six samples may be tested simultaneously in the remaining wells 1. In the illustrated experiment, the device is testing for the presence of SARS-CoV-2 in oral-nasal samples, using the assay described above.

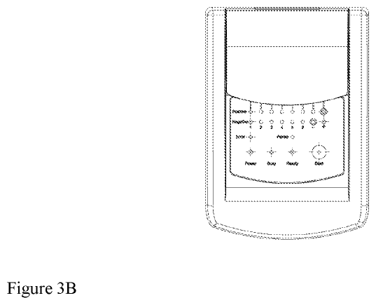

Once the sample tubes are loaded, the user presses a “start” button 5 on the device (as seen in FIGS. 3A&B). The sample tubes are heating to between about 62° C. and 68° C. for approximately 30 minutes. 8 blue LEDs, each aligned with a sample well, emit light at 470 nm, which excites fluorescent dye SYTO-9, the concentration of which increases over time when the LAMP reaction occurs.

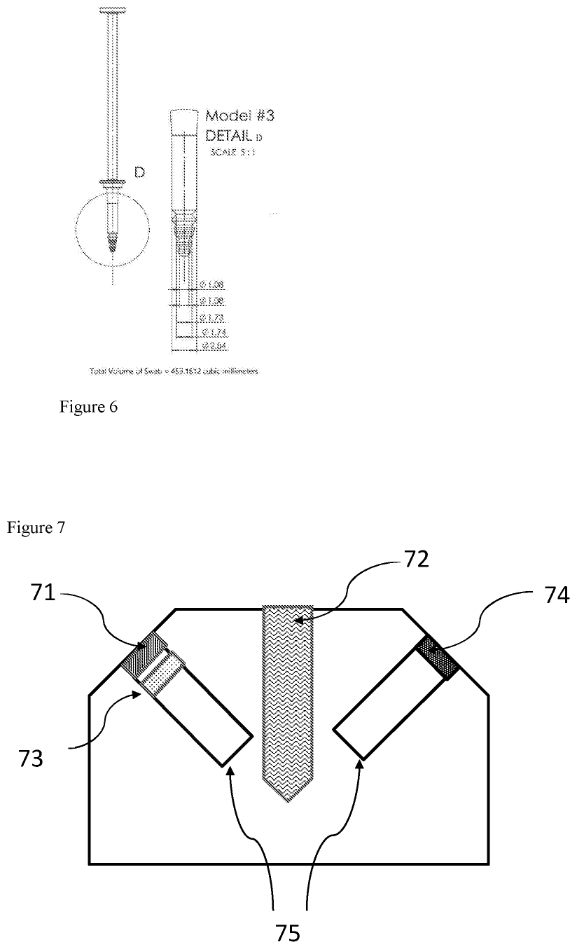

An optical fibre is aligned with each sample well to transmit fluorescent emission to photodiodes. The optical filter extends away from the sample well at 90° from the angle of incident LED light entering the sample well, so that there is no line-of-sight between the LED and photodiode. An optical filter is also provided between the sample well and photodiode, which filters the excitation wavelength 470 nm. (The fluorescent emission has a wavelength of around 520 nm.) With reference to FIG. 7, it can be seen that the incident light from the LED 74 passes along an optical fibre to the sample well 72. From the sample well it passes along an optical fibre 75 arranged perpendicularly to the first optical fibre, through an optical filter 73 and to the photodiode 74.

The fluorescence is measured every 10 seconds by the device. An integrated algorithm, based on that illustrated in FIG. 1, processes the fluorescence data and classifies whether the sample is positive (i.e. contains target nucleic acid), negative (does not contain target nucleic acid) or undetermined and this is communicated to the user via an LED user interface 7. The user interface comprises two LEDs for each sample well; a red LED indicates that the sample is positive for the target nucleic acid, a green LED indicates a negative sample, and where the algorithm classifies “undetermined”, both LEDs illuminate. In FIG. 3A, the first sample (furthest left) is positive, samples 2 to 6 are negative, the seventh well contains the negative control and the eighth well (furthest right) contains the positive control.

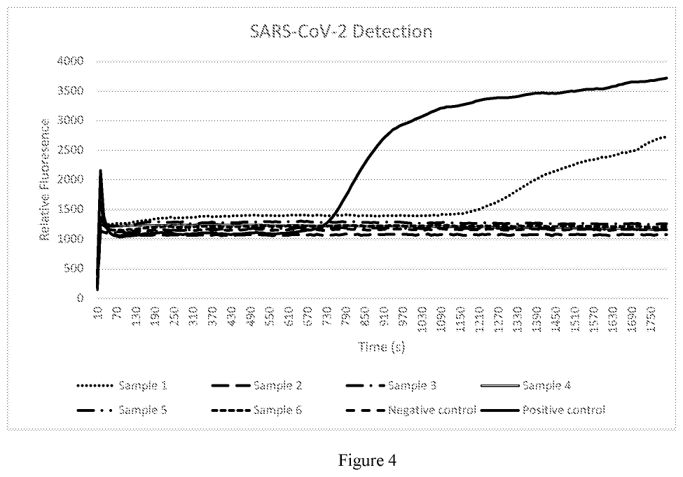

The detected fluorescence for each of the 8 wells is shown in FIG. 4. The y axis shows relative fluorescence and is calibrated for the device, where 0 corresponds to no fluorescence detected, and 4000 corresponds to detector saturation. For this device and assay combination, the algorithm parameters (for the algorithm illustrated in FIG. 1) are:

$$\begin{matrix}{C}_{1} = 1800 ( where {C}_{1} is mean value between time = 120 seconds and time = 300 seconds ) \\ {C}_{2} = 18 * detected baseline fluorescence \\ {C}_{3} = 480 ( seconds )\end{matrix}$$

The samples in this experiment were also subjected to comparative PCR testing (according to standard UK PCR Covid-19 testing protocol), and the determination matched the PCR output for all samples.

FIG. 5 shows further experimental data, illustrating how false positives may be avoided. In this case, it can be seen that increases in fluorescence are observed for samples 7, 8, and 11. However, the fluorescence level for sample 7 begins to rise too early, and the algorithm returns an “undetermined” outcome. For sample 8, the baseline level is too high, and the algorithm returns an “undetermined” outcome. For sample 11, the peak fluorescence is too low, and the algorithm returns a “negative” outcome. The samples in this experiment were also subjected to comparative PCR testing and all were “negative” in the PCR output (apart from the positive control). It can thus be seen that the inventive method described herein has avoided returning erroneous positive outcomes for samples 7, 8 and 11.

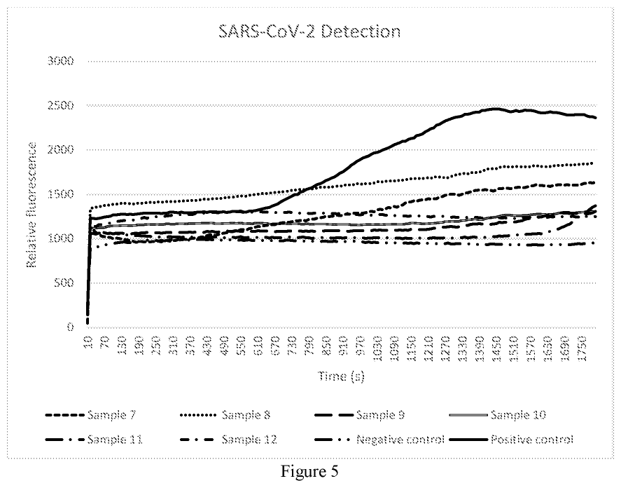

Oral-nasal samples tested in the description above are collected using a swab, which is illustrated in FIG. 6. The swab is a hard, clean medical grade polypropylene stick with ribbed structure 9 at the tip to collect the sample. The tip is connected to a handle 11 at the opposite end, and a collar 13 at an intermediate point along the swab length separates the tip and handle.

The ribbed structure 9 has 4 ribs and collects a reproducible volume of liquid by adhesive and capillary action; in this instance, the swab collects 0.45 ml±0.05 ml. The swab design allows for subsequent elution of the sample into the reaction mixture in a standard 200 μL PCR tube. As can be seen in FIG. 6, the diameter of the ribs in the rib structure narrows towards to tip end, mirroring the profile of a standard PCR tube. The widest ribs (nearest to the collar) are 2.84 mm in diameter, and the narrowest (at the tip end) at 1.08 mm in diameter. The narrowing tip also improves patient comfort and safety.

The collar 13 may serve as a lid for a standard 200 μL PCR tube during sample elution. The tip length is shorter than the length of the PCR tube (i.e. shorter than 20.1 mm). The collar diameter is 6.3 mm (i.e. wider than the PCT tube opening).

In some cases, the swab is removed from the PCR tube after elution and before analysis, in which case, another lid (typically rubber) may be used to seal the tube.

In other cases, the swab tip may remain in the PCR tube during analysis. In such cases, the handle is typically snapped off after insertion of the tip into the tube. The swab material (polypropylene) is the identical to the material forming standard 200 μL PCR tubes, and thus does not interfere with the reaction. Furthermore, polypropylene has low fluorescence around blue light (˜470 nm wavelength) and so does not affect the fluorescence readings. Finally, the swab has approximately infinite rotational symmetry, and so the orientation at insertion is not at issue (in contrast to an asymmetric swab, for example, where differing refraction effects depending on orientation on insertion into the tube may cause inconsistent fluorescence readings).

The above embodiments are to be understood as illustrative examples of the invention. Further embodiments of the invention are envisaged. It is to be understood that any feature described in relation to any one embodiment may be used alone, or in combination with other features described, and may also be used in combination with one or more features of any other of the embodiments, or any combination of any other of the embodiments. Furthermore, equivalents and modifications not described above may also be employed without departing from the scope of the invention, which is defined in the accompanying claims.

1. A method of detecting a target nucleic acid in a biological sample, the method comprising:
i) combining the sample with reagents to enable amplification of the target nucleic acid and fluorescent labelling of any amplified product;
ii) detecting fluorescence, and optionally determining that the baseline fluorescence level of the amplification reaction does not exceed a predetermined threshold; and
iii) determining whether the target nucleic acid is present in the sample based on the following parameters:
a) time to start of reaction, signalled by an increase in fluorescence, wherein the method involves monitoring fluorescence over time to establish when fluorescence starts to increase relative to the time when the sample and reagents are combined, and wherein if an increase in fluorescence starts outside of a defined time window, no determination is possible as to whether the target nucleic acid is present; and
b) peak fluorescence intensity, wherein if the peak fluorescence intensity does not exceed a predetermined threshold, the determination is that the target nucleic acid is absent.

2. (canceled)

3. A method according to claim 1, wherein the method includes determining that the baseline fluorescence level of the amplification reaction does not exceed a predetermined threshold.

4. A method according to claim 1, wherein if the baseline fluorescence level exceeds the predetermined threshold, no determination is possible as to whether the target nucleic acid is present.

5. (canceled)

6. (canceled)

7. A method according to claim 1, wherein the nucleic acid in the biological sample is DNA or RNA.

8. A method according to claim 7, wherein when the nucleic acid in the biological sample is RNA, the sample is contacted with reverse transcriptase to generate a double stranded target nucleic acid for amplification.

9. A method according to claim 1, wherein the reagents effect an isothermal amplification reaction.

10. A method according to claim 1, wherein the reaction is a loop-mediated isothermal amplification (LAMP) and the method comprises contacting the sample with a set of LAMP primers specific for the target nucleic acid.

11. A method according to claim 1, wherein the target nucleic acid is from a microorganism.

12. A method according to claim 11, wherein the microorganism is a bacterium.

13. A method according to claim 12, wherein the bacterium is Escherichia coli, Clostridium or Streptococcus.

14. A method according to claim 11, wherein the microorganism is a virus.

15. A method according to claim 14, wherein the virus is selected from the group consisting of an influenza virus, a coronavirus, respiratory syncytial virus, adenovirus, varicella zoster, cytomegalovirus and Hepatoviruses.

16. A method according to claim 11, wherein the method detects the presence of absence of SARS-CoV-2.

17. A method according to claim 16, wherein the amplification is a LAMP reaction and wherein the set of LAMP primers comprises six primers to specifically amplify the E1 and/or N2 targets in the SARS-CoV-2 RNA genome.

18. A method according to claim 17 wherein the set of LAMP primers comprises six primers for the detection of E1 having nucleic acid sequence at least 95% identical to SEQ ID NOs: 1-6.

19. A method according to claim 17 wherein the set of LAMP primers comprises six primers for the detection of N2 having nucleic acid sequence at least 95% identical to SEQ ID NOs: 7-12.

20. A method according to claim 17, wherein the reaction mixture also comprises guanidine hydrochloride.

21. (canceled)

22. (canceled)

23. A device for detecting a target nucleic acid in a biological sample, the device comprising;
a reaction chamber, which in use receives the sample and reagents for performing amplification and fluorescent labelling of the amplification product;
a light source which emits light at a wavelength corresponding to an excitation wavelength of the fluorescent label;
a detector, for detecting fluorescence; and
a user interface;
wherein the device is programmed to classify the biological sample as positive, negative or undetermined for the presence of the target nucleic and present the classification on the user interface, the classification being based on:
a) time to start of reaction, signalled by an increase in fluorescence, wherein the device monitors fluorescence over time to establish when fluorescence starts to increase relative to the time when the sample and reagents are combined, and wherein if an increase in fluorescence starts outside of a defined time window, the device classifies the sample as undetermined; and
b) peak fluorescence intensity, wherein if the peak fluorescence intensity does not exceed a predetermined threshold, the device classifies the sample as negative;
c) and optionally baseline fluorescence level, wherein if the baseline fluorescence level exceeds the predetermined threshold, the device classifies the sample as undetermined.

24. A device for detecting a target nucleic acid from a microorganism in a biological sample, the device comprising;
a reaction chamber, which in use receives the sample and reagents for performing amplification and fluorescent labelling of the amplification product;
a light source which emits light at a wavelength corresponding to an excitation wavelength of the fluorescent label;
a detector, for detecting fluorescence; and
a user interface, which indicates whether the target nucleic acid is present in the sample; wherein the device further comprises a filter between the reaction chamber and detector, which filters light at wavelengths emitted by the light source, and wherein there is no direct line of sight between the light source and detector.

25. A device according to claim 24, wherein the reaction chamber comprises a well in a heating block, and wherein an insulative layer at least partially surrounds the heating block.

26. (canceled)

27. (canceled)

28. (canceled)

29. (canceled)

30. (canceled)

31. (canceled)


SEQUENCE LISTING (12 sequences)

SEQ ID NO: 1 - DNA, 23 bp, Artificial Sequence
  Primer
tgagtacgaacttatgtactcat

SEQ ID NO: 2 - DNA, 22 bp, Artificial Sequence
  Primer
ttcagatttttaacacgagagt

SEQ ID NO: 3 - DNA, 42 bp, Artificial Sequence
  Primer
accacgaaagcaagaaaaagaagttcgtttcggaagagacag

SEQ ID NO: 4 - DNA, 44 bp, Artificial Sequence
  Primer
ttgctagttacactagccatccttaggttttacaagactcacgt

SEQ ID NO: 5 - DNA, 18 bp, Artificial Sequence
  Primer
cgctattaactattaacg

SEQ ID NO: 6 - DNA, 19 bp, Artificial Sequence
  Primer
gcgcttcgattgtgtgcgt

SEQ ID NO: 7 - DNA, 21 bp, Artificial Sequence
  Primer
accaggaactaatcagacaag

SEQ ID NO: 8 - DNA, 25 bp, Artificial Sequence
  Primer
gacttgatctttgaaatttggatct

SEQ ID NO: 9 - DNA, 42 bp, Artificial Sequence
  Primer
ttccgaagaacgctgaagcggaactgattacaaacattggcc

SEQ ID NO: 10 - DNA, 40 bp, Artificial Sequence
  Primer
cgcattggcatggaagtcacaatttgatggcacctgtgta

SEQ ID NO: 11 - DNA, 21 bp, Artificial Sequence
  Primer
gggggcaaattgtgcaatttg

SEQ ID NO: 12 - DNA, 20 bp, Artificial Sequence
  Primer
cttcgggaacgtggttgacc